(19) United States
(12) Patent Application Publication
(10) Pub. No.: US 2020/0262785 A1
(43) Pub. Date: Aug. 20, 2020

(54) 2-CYANO-2-FLUOROETHENOLATE SALTS (CFES): VERSITILE ACTIVE PHARMACEUTICAL INTERMEDIATES

(71) Applicant: Till Opatz, Oberursel (DE)

(72) Inventor: Till Opatz, Oberursel (DE)

(21) Appl. No.: 16/279,501

(22) Filed: Feb. 19, 2019

(51) Int. Cl. C07C 255/19

(52) U.S. Cl. CPC C07C 255/19

(57) ABSTRACT

The present invention relates to new enolate structures with utility as active pharmaceutical intermediates for the preparation of efficacious drugs such as those derived from 5-fluorocytosine (5-FC).

### FIELD OF THE INVENTION

2-Cyano-2-fluoroethenolate salts (CFES) and a process for making them are disclosed which compounds are of,

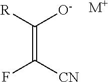

wherein M⁺ is a stable, non-reactive, cation of the alkali metal family or related stable cations compatible with the subject enolate anion and the desired subsequent reaction conditions for its use, and R is hydrogen, hydrocarbyl, or substituted hydrocarbyl. CFES may be used as an active pharmaceutical intermediate to obtain valuable and efficacious drugs such as those derived from 5-fluorocytosine (5-FC). CFES may be obtained through a Claisen-type condensation from fluoroacetonitrile.

The term “hydrocarbyl” is used herein to mean any substituent group that is composed of only carbon and hydrogen atoms in any connectivity (linear or branched), cyclicity (cyclic or acyclic), chirality (and achiral), saturated or unsaturated. The term “hydrocarbyl” thus may include alkyl, aryl, olefinic, or acetylenic moieties, alone or in combination. The term “substituted hydrocarbyl” is used herein to mean a hydrocarbyl moiety that bears any substituent group which does intra- or inter-molecularly react with another portion of the CFES enolate salt, or another component of a reaction process in which CFES is subsequently employed leading to unwanted side-reactions. For example suitable substituents may include, but are not limited to, cyano, ether, halo, vinyl, and substituted imidazoles.

### BACKGROUND OF THE INVENTION

Though a variety of separately cyano- or fluoro-substituted enolates are reported in the chemical literature, the combination of a cyanofluoroenolate composition is unknown in the chemical literature. The availability of CFES would have a substantially positive impact on the price and availability of a number of pharmaceuticals. There is an urgent demand for the drug 5-fluorocytosine (5-FC) in low-income countries due to its activity against HIV-induced fungal infections as well as its use as a key intermediate in the synthesis of the clinically highly important anti-HIV drug emtricitabine (FTC). More particularly, the present invention is directed to a novel class of fluorocyanoenolates with application in facile production of therapeutic drugs and other pharmaceutical intermediates. There are only a few 5-fluorocytosine syntheses available and most of them use an electrophilic fluorination with the highly expensive and dangerous gaseous fluorine. [Harsanyi et al. Org. Process Res. Dev., 2017, 21(2), pp 273-27; Baasner et al. U.S. Pat. No. 4,703,121, October 1987; Griller et al. U.S. Pat. No. 3,846,429 November 1974] Handling of fluorine gas on an industrial scale poses additional problems for the manufacture of pharmaceuticals. The present invention with the use of CFES as an active pharmaceutical intermediate allows for a safe, easy, and inexpensive nucleophilic fluorination at an earlier synthetic stage. A different early-stage F-introduction strategy was developed by Hoffmann-La Roche, Inc. [Duschinsky et al. J. Am. Chem. Soc., 1957, 79(16), pp 4559-4560] However, this latter approach furnishes 5-FC in only 11% yield over five steps, and requires the use of relatively expensive S-ethylisothiouronium bromide.

### BRIEF DESCRIPTION OF THE DRAWING

FIG. 1 shows the chemical formula for the cyanofluoroenolate salts (CFES) described in this invention. The atom connectivity, but not the double bond geometry ((E)- or (Z)-isomeric form), of the CFES is specified by this drawing.

### DETAILED DESCRIPTION OF THE INVENTION

### 2-Cyano-2-fluoroethenolate Salts (CFES)

The present invention relates to new enolate structures with utility as active pharmaceutical intermediates for the preparation of efficacous drugs such as those derived from 5-fluorocytosine (5-FC).

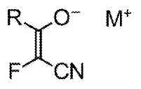

According to the present invention there is provided a new class of substituted enolates of the formula:

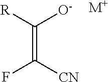

wherein M⁺ is a stable, non-reactive, cation, and R is hydrogen, hydrocarbyl, or substituted hydrocarbyl (FIG. 1).

CFES may be obtained through a Claisen-type condensation from fluoroacetonitrile (Example 1). A simple and low-cost 2-step synthesis of 5-FC starting from CFES (Example 2) provides an example of the utility of CFES. An overall yield up to 82% could be achieved for 5-FC using the new CFES composition and the route is devoid of any chromatographic purifications. This new route made possible by the CFES composition is lower cost and more efficient than presently available methods.

### Example 1

### Potassium (Z)-2-cyano-2-fluoroethenolate

In a flame-dried Schlenk flask, KO^{t}Bu (9.1 g, 81 mmol, 1.5 eq.) was dissolved in dry tetrahydrofuran (THF) (26 mL) under argon atmosphere. The solution was cooled to −15° C. using a cryostat. In a second flame-dried Schlenk flask, a solution containing fluoroacetonitrile (3.0 mL, 54 mmol, 1.0 eq.), ethyl formate (17.4 mL, 21.6 mmol, 4.0 eq.) and dry THF (30 mL) was prepared under argon atmosphere and cooled to −15° C. By using a syringe pump the KOtBu/THF solution was added dropwise to the fluoroacetonitrile/ethyl formate/THF solution at a rate of 1 mL/min while stirring vigorously. After the addition was complete, the reaction mixture was cooled for a further 20 min. The colorless suspension was stirred overnight (20 h) at room temperature. Afterwards, n-hexane (30 mL) was added and the slightly brown suspension was cooled in an ice bath. After 5 min, the precipitate was filtered off and washed with cold n-hexane. The obtained solid was dried in a vacuum desiccator to yield a slight brownish solid (6.21 g) which contains (Z)-2-cyano-2-fluoroethenolate (5.17 g 41.3 mmol, 77%, estimated by ¹H NMR) and potassium formate. The yield was corrected for the content of potassium formate. Tₘ=91° C. (decomposition). ¹H-NMR (300 MHz, DMSO-d₆): δ=7.48 (d, ³J_{H-F}=30.5 Hz, 1H, H-1) ppm. ¹³C-NMR, HSQC, HMBC (75 MHz, DMSO-d₆): δ=157.9 (d, ³J_{C-F}=9.0 Hz, C-1), 124.2 (²J_{C-F}=33.5 Hz, CN), 120.5 (d, ¹J_{C-F}=196.5 Hz, C-2) ppm. ¹⁹F-NMR (282 MHz, DMSO-d₆): δ=−207.1 (d, ³J_{F-H}=30.5 Hz) ppm. IR (ATR): v=2180, 1593, 1350, 1323, 1206 cm⁻¹. ESI-HRMS: Calcd for C₃HFKNO ([M]⁻): m/z=86.0046, Found: m/z=86.0046.

### Example 2

### 5-Fluoropyrimidine-2,4-diamine

In a flame-dried Schlenk flask, guanidine carbonate (2.22 g, 24.7 mmol, 3.0 eq.) was dissolved in dry methanol (18 mL) under argon atmosphere. A 5.4M methanolic sodium methoxide solution (4.7 mL, 26 mmol, 3.1 Eq.) was dripped quickly into the solution, before potassium (Z)-2-cyano-2-fluoroethenolate (1.03 g, 8.23 mmol, 1.0 eq.) was added portionwise over 5 min. The brownish suspension was stirred at rt for 18 h. The solvent was evaporated at 40° C. under reduced pressure and the residue was dissolved in water (20 mL). The aqueous suspension was extracted with ethyl acetate (4×20 mL). The combined organic phases were dried over sodium sulfate and the solvent was evaporated in vacuo. 5-fluoropyrimidine-2,4-diamine (0.96 g, 7.5 mmol, 91%) was obtained as a colorless to slight brownish powder. Tₘ=157.6-160.8° C. TLC (SiO₂): R_{f}=0.30 (ethyl acetate:MeOH=20:1). ¹H-NMR (300 MHz, DMSO-d₆): δ=7.65 (d, ³J_{H-F}=3.9 Hz, 1H, H-6), 6.64 (s_{b}, 2H, NH₂), 5.81 (s_{b}, 2H, NH₂) ppm. ¹³C-NMR, HMBC, HSQC (75 MHz, DMSO-d₆): δ=159.8 (d, ⁴J_{C-F}=3.0 Hz, C-2), 153.6 (²J_{C-F}=12.3 Hz, C-4), 140.0 (d, ¹J_{C-F}=239.5 Hz, C-5), 139.9 (d, ²J_{C-F}=18.4 Hz, C-6) ppm. ¹⁹F-NMR (282 MHz, DMSO-d₆): δ=−171.2 (d, ³J_{F-H}=3.9 Hz) ppm. IR (ATR): v=3408, 3330, 3139, 1671, 1590, 1442, 1213 cm⁻¹. ESI-HRMS: Calcd for C₄H₅FN₄ ([M+H]⁺): m/z=129.0571, Found: m/z=129.0570.

### 5-Fluorocytosine

In a round bottom flask, 5-fluoropyrimidine-2,4-diamine (4.32 g, 33.7 mmol, 1.0 eq.) was dissolved in water (100 mL) and cooled in an ice-bath. Precooled half-concentrated sulfuric acid (18.7 mL, 84.3 mmol, 2.5 eq.) was added slowly. While keeping the reaction temperature at 1-3° C., a 2.5N sodium nitrite solution (5.82 g NaNO₂ in 34 mL water, 84.3 mmol, 2.5 eq.) added within 17 min. The reaction mixture was stirred for another 20 min while cooling and 1 h at rt. The clear yellow solution was adjusted to pH=7.4 by adding conc. ammonia solution (20 mL, 25 wt %) whereby a slightly orange suspension was formed. The mixture was concentrated in vacuo at 40° C. to half of its original volume and cooled in an ice-bath. The precipitate was collected by suction filtration and washed with small portions of ice water (3×4 mL). After drying the solid in air and in a desiccator over molecular sieves, 5-fluorocytosine (3.61 g, 28.0 mmol, 83%) was obtained as a colorless to slightly brown powder. Tₘ=295° C. (decomposition) (Lit.: 295-300° C., decomposition [Harsanyi et al. Org. Process Res. Dev., 2017, 21(2), pp 273-27]). TLC (SiO₂): R_{f}=0.29 (ethyl acetate:MeOH=3:1). ¹H-NMR (300 MHz, DMSO-d₆): δ=7.60 (d, ³J_{H-F}=6.2 Hz, 1H, H-6), 7.34 (s_{b}, 1H, NH) ppm. ¹³C-NMR, HMBC, HSQC (75 MHz, DMSO-d₆): δ=158.2 (d, ²J_{C-F}=13.0 Hz, C-4), 155.3 (s, C-2), 136.0 (d, ¹J_{C-F}=237.9 Hz, C-5), 127.0 (d, ²J_{C-F}=29.3 Hz, C-6) ppm. ¹⁹F-NMR (282 MHz, DMSO-d₆): δ=−171.6 (d, ³J_{F-H}=6.2 Hz) ppm. IR (ATR): v=3337, 3126, 2724, 1678, 1542, 1460, 1227, 1123 cm⁻¹. ESI-MS: m/z=130.1 (100%, [M+H]⁺). The spectroscopic data are consistent with literature values. [Harsanyi et al. Org. Process Res. Dev., 2017, 21(2), pp 273-27]

1-8. (canceled)

9. A compound of formula: 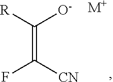
wherein the compound is isolated in solid form, M⁺ is an alkali metal cation, and R is hydrocarbyl, or substituted hydrocarbyl.

10. The compound of claim 9, wherein M is potassium (K).

11. The compound of claim 9, wherein M is sodium (Na).

12. The compound of claim 9, wherein M is lithium (Li).

13. The compound of claim 9, wherein R is hydrocarbyl.

14. The compound of claim 9, wherein R is substituted hydrocarbyl.

15. The compound of claim 9, wherein M is selected from the group consisting of Li, Na, K, Rb, and Cs.

16. A compound of formula: 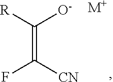
wherein M⁺ is an alkali metal cation, and R is hydrogen.

17. The compound of claim 16, wherein M is potassium (K).

18. The compound of claim 16, wherein M is sodium (Na).

19. The compound of claim 16, wherein M is lithium (Li).

20. The compound of claim 16, wherein M is selected from the group consisting of Li, Na, K, Rb, and Cs.
